(19) United States
(12) Patent Application Publication
(10) Pub. No.: US 2024/0352512 A1
(43) Pub. Date: Oct. 24, 2024

(54) DETECTION OF VARIANTS

(71) Applicant: Saga Diagnostics AB, Lund (SE)

(72) Inventors: Miguel Alcaide, Lund (SE); Anthony Miles George, Lund (SE); Yilun Chen, Lund (SE); Lucia Oton, Malmö (SE)

(21) Appl. No.: 18/644,268

(22) Filed: Apr. 24, 2024

Related U.S. Application Data
(60) Provisional application No. 63/497,872, filed on Apr. 24, 2023.

(51) Int. Cl. C12Q 1/6858

(52) U.S. Cl. CPC C12Q 1/6858

(57) ABSTRACT

The invention provides a method that includes performing a pre-amplification of nucleic acid in a sample to generate amplicons that include a copy of a variant, annealing and extending variant-specific primers to create copies of the amplicon, and copying the copies with tailed primers to form tailed amplicons. The tailed amplicons are amplified with universal primers and detection probes that anneal to the tailed amplicons. Extension of the universal primers along the tailed amplicons through annealed detection probes generates a signal that shows the presence of the variant in the sample.

### TECHNICAL FIELD

The disclosure relates to the detection of variants nucleic acids.

### BACKGROUND

Detection of nucleic acids present in samples at relatively low quantities is desirable for many clinical applications. For example, it is recognized that exceptionally sensitive and specific methods for mutation detection are necessary, in particular for low-input samples such as circulating cell-free DNA (cfDNA). Conventional methods seek to capture and amplify nucleic acids of interest using the polymerase chain-reaction (PCR).

Digital PCR (dPCR) is a method that involves dividing (or “partitioning”) a sample into large number of small reaction volumes and conducting PCR in each partition to detect if a nucleic acid of interest is present. The distribution of nucleic acids into reaction volumes is random and is understood to follow a Poisson distribution. In effect, each nucleic acid molecule can be isolated in its own partition allowing each molecule to be individually detected. The result is a potential increase in sensitivity. However, dPCR does not always detect the presence of low-abundance target nucleic acid in a biological sample. Thus, existing dPCR approaches are not always adequate for the detection of clinically-significant variants, such as structural variants, that may indicate the presence of a disease.

### SUMMARY

The present invention provides methods for detecting low-abundance nucleic acids, including mutations, such as structural variants. The invention uses selective pre-amplification to increase the abundance of a target of interest in a sample, even when that target is present in very low numbers. The invention solves specific problems with molecular detection assays that rely on PCR. For example, due to the stochastic nature of PCR low-abundance targets may go undetected if not amplified in early rounds of PCR. Also, conventional dPCR workflows suffer from significant “dead volume”, which is the fraction of sample that is consumed but never analyzed leading to loss of sensitivity. Those problems are addressed by selectively amplifying a target of interest in what is described here as a pre-amplification step. Rare targets of interest are increased in abundance by this pre-amplification step. The pre-amplification of nucleic acid in a sample generates amplicons that include a copy of a variant of interest. Preferably, the pre-amplification is performed on the sample prior to partitioning for digital PCR (dPCR). The pre-amplification uses PCR reagents with primers designed to flank a variant of interest, increasing the abundance of copies (e.g., amplicons) that include that variant.

Once pre-amplification has been performed, the sample is partitioned and subject to a dPCR protocol that involves two stages, or types, of amplification. In the first stage of the dPCR, the amplicons are copied using variant-specific primers and tailed primers that operate to form tailed amplicons, which are then probed by dPCR. Methods of the invention have high sensitivity and are useful for discovering structural variants (SV) in samples including, for example, a tumor-specific structural variants in cfDNA in a blood or plasma sample. Due to that sensitivity, methods of the invention provide a useful and easy method for the detection of minimal residual disease after treatment. The variant-specific primer may preferably be a breakpoint-spanning primer that anneals substantially on one side of a breakpoint of a structural variant, but has at least a few bases at the 3′ end that anneal on the other side of the breakpoint. By using a breakpoint spanning primer, dPCR is expected, in the majority of situations, to only give a positive result for aqueous partitions that include a copy of the SV. Methods of the invention comprise a linear amplification of the SV via extension of the breakpoint-spanning primer at high annealing temperature; and an exponential amplification of the SV with the participation of a tailed primer and the universal primer at a lower annealing temperature. Annealing and hydrolysis of probes, which translates in the emission of fluorescent signal, also happens during the exponential phase.

In certain embodiments, methods include amplifying the tailed amplicons with universal primers and detection probes that anneal to the tailed amplicons. Extension of the universal primers along the tailed amplicons through annealed detection probes generates a signal that shows the presence of the variant in the sample. For example, the probes may be fluorescently quenched hydrolysis probes that are digested by a 5′-3′ exonuclease activity of the polymerase. Thus, aqueous partitions that include amplicons with copies of the variant of interest will produce unquenched fluorophores, and will fluoresce. The dPCR reaction volumes can be monitored by a fluorescence detector, a microscope, or other dPCR instrument to detect presence of the variant in the sample.

In a certain aspect, the invention provides methods that include performing pre-amplification of nucleic acid in a sample to generate amplicons that include a copy of a variant, annealing and extending variant-specific primers to create copies of the amplicon, and copying the copies with tailed primers to form tailed amplicons. The tailed amplicons are amplified with universal primers and detection probes that anneal to the tailed amplicons. Extension of the universal primers along the tailed amplicons through annealed detection probes generates a signal that shows the presence of the variant in the sample. The sample may be a blood or plasma sample and the detected nucleic acid may be cell-free DNA (cfDNA) in the blood or plasma. In some embodiments, the variant is a structural variant (SV) and the pre-amplification is performed with a PCR primer pair designed to anneal to sites that flank a breakpoint of the structural variant. Some methods may include partitioning the samples into aqueous compartments (e.g., droplets, microchambers, or wells of a plate) after the pre-amplification and performing the amplifying step as digital PCR in the aqueous compartments.

After a pre-amplification, the sample may be partitioned and a first amplification performed with variant specific primers and tailed primers. The variant-specific primers may be breakpoint-spanning primers. The tailed primers generate tailed amplicons. The annealing and extending of the tailed primers and variant-specific primers may include thermocycling the amplicons in the presence of the variant-specific primers at temperatures that yields substantially only linear production of the copies. The variant-specific primers may be designed to anncal near, and be extended through, a variant of interest. Preferably, the variant-specific primers are designed to anneal to, and only be extended in the presence of, a variant of interest. The variant may be a polymorphism, a small indel, or a breakpoint of a structural variant.

The downstream amplifying steps may be performed together by thermocycling at temperatures that promote exponential amplification. For detection, a second amplification is performed on the tailed amplicons in the presence of a detection probe. The detection probe may be a hydrolysis probe that anneals to the tail and is digested by exonuclease activity of a polymerase used for the amplifying step. The detection probes may be universal probes that anneal to a universal probe binding site on a tail of the tailed amplicons.

In certain embodiments, the method includes inactivating a polymerase enzyme in the sample after the pre-amplification. The inactivating step may be performed with a thermolabile proteinase and the method may include heat denaturing the proteinase prior to the dPCR amplifying steps. In some embodiments, no sample cleanup is performed between steps. The amplifying step may be performed in the presence of residual primers and reagents from the pre-amplification.

Steps of the methods described herein may be performed with multiplexed primer and probe sets that amplify and detect multiple distinct variants of interest. The detection probes may include one or more universal primer that anneals to a universal binding site on a tail of the tailed amplicons. In some embodiments, identical copies of the universal probe can be used simultaneously in a single dPCR assay to detect the multiple distinct variants of interest.

The described method may be performed in parallel with a reference assay. The steps of the method may be performed with reference primer and probe sets that amplify and detect copy number stable loci to produce a reference signal and the method includes estimating genomic equivalents in the sample from the reference signal.

In a further instance of the invention, multiple tailed primers are used in a single reaction where the universal probe segment is distinct between tailed primers but the universal primer segment is common. So a single universal primer generates signal in multiple channels.

### BRIEF DESCRIPTION OF THE DRAWINGS

FIG. 1 diagrams steps of a variant detection method.

FIG. 2 illustrates elements used in the pre-amplification.

FIG. 3 illustrates elements used in forming tailed amplicons.

FIG. 4 shows detection of the target.

### DETAILED DESCRIPTION

The disclosure provides systems and methods useful for the multi-channel, ultra-sensitive detection of pre-amplified targets of interest such as structural variants. In general, methods of the invention include (i) performing a nucleic acid pre-amplification reaction on a sample to pre-amplify one or more variants of interest in the sample, and then subjecting the product of the pre-amplification to detection by a method such as digital PCR. The detection stage may include partitioning the sample into aqueous compartments, such as wells in a plate or droplets. Each compartment may be provided with reagents for an amplification reaction that yields a detectable signal when the target of interest is present. In preferred embodiments, the detection may proceed by at least two distinct stages or mechanisms that include (ii) copying pre-amplification products using variant-specific primers and tailed primers to form tailed amplicons and (iii) amplifying the tailed amplicons in the presence of probes that indicate the presence of amplicons from the target of interest in the aqueous compartment.

The (i) pre-amplification step may use primers designed to specifically amplify nucleic acid suspected of harboring a variant of interest. For example, if the variant is a structural variant, the pre-amplification may use a pair of primers designed to anneal to nucleic acid at locations that flank a breakpoint of the structural variant. This strategy enriches the sample for copies of the variant, ensuring that the presence of the variant is detected in the subsequent detection steps. This pre-amplification step specifically addresses problems associated with some dead volume of sample that resists detection by existing digital PCR (dPCR) approaches. Due to the stochastic nature of sampling, some very minor fraction of a sample will, by chance, typically go undetected by dPCR. Here, the pre-amplification step specifically increases quantity of the target of interest prior to the partitioning and dPCR detection, reducing the likelihood that the target of interest will be undetected due to stochastic loss in dead volume. After the pre-amplification, the sample may be partitioned into aqueous compartments.

Once partitioned, the (ii) copying step uses variant-specific primers and tailed primers. The variant-specific primers are preferably designed to anneal to, and only prime synthesis of copies of, nucleic acids that harbor the variant of interest. For example, where the variant of interest is an SV, the variant specific primer may predominantly sit adjacent a breakpoint of the SV, but have a few bases at a 3′ end that extend pass the breakpoint. That is, the primer anneals across the breakpoint of the SV. This primer reduces the likelihood of non-specific amplification (i.e., reduces noise). The tailed primer may include either or both of a universal primer binding site and a universal probe binding site. Copying with the variant-specific primers and the tailed primers yields tailed amplicons that specifically include copies of the variant and universal primer and probe binding sites.

The tailed amplicons are (iii) amplified in the presence of detection probes, such as fluorescent hydrolysis probes. The detection probes are preferably “universal” probes in that they anneal to universal probe binding sites in the tailed amplicons and do not depend on the presence a specific genetic sequence from within the genomic information of the target. This final amplification and detection step preferably also includes universal primers that, similarly, anneal to universal primer binding sites in the tailed amplicons and do not depend on the presence a specific genetic sequence. The use of the tailed primer to form the tailed amplicons allows the detection stage of the dPCR workflow to operate with universal primers and probes, meaning that the (i) pre-amplification and (ii) copying with tailed primer steps can be customized to target organism, patient, mutation, melting temperature, or other such factor, while the (iii) amplification and detection does not require the synthesis of custom reagents.

The disclosed methods are useful for detection of any suitable target of interest in a sample. For example, methods are useful to detect nucleic acid from a pathogen in a mixed environmental sample or in a clinical sample that includes abundant host nucleic acid. The method may be used to detect fetal DNA in maternal blood or plasma. In certain preferred embodiments, the method is useful to detect a variant associated with a disease, such as an SV from tumor DNA, in cell-free DNA in a sample from a patient.

Any suitable sample may be used. For example, the sample may be blood, saliva, solid tissue, fine needle aspirate, a tumor biopsy (including material liberated from a formalin-fixed, paraffin embedded tumor sample), oral (e.g., buccal) swab, urine, stool, or any other sample. In certain embodiments, the sample comprises blood or plasma and the nucleic acid comprises cell-free DNA (cfDNA) in the blood or plasma.

Methods of the invention may be used to detect any nucleic acid feature of interest including, for example, specific sequences, genes, or variants (e.g., mutations), which may include polymorphisms, small indels, or structural variants (which may include deletions, rearrangements, large indels, translocations, copy number variants, or others). In certain embodiments, the variant is a structural variant (SV) and a pre-amplification is performed with a PCR primer pair design to anneal to sites that flank a breakpoint of the SV. Preferred embodiments provide methods useful to detect nucleic acid fragments containing targeted structural variants present in a sample at low abundance (e.g., as low as one copy).

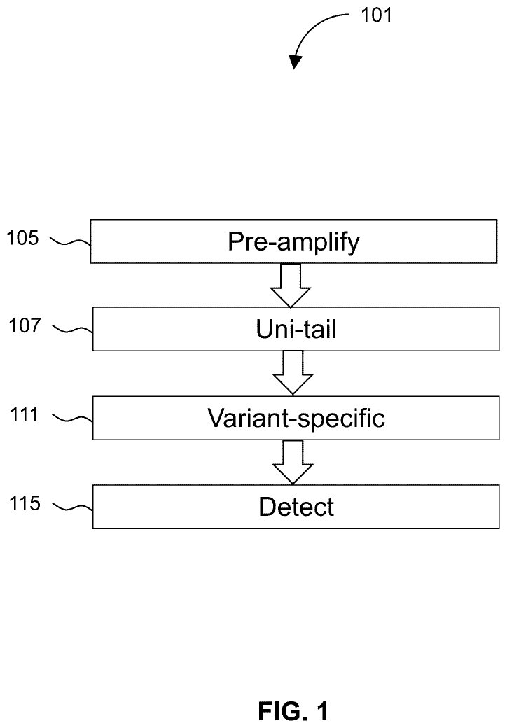

FIG. 1 diagrams steps of a variant detection method 101. The method 101 includes performing a pre-amplification 105 of nucleic acid in a sample to generate amplicons that include a copy of a variant. The pre-amplification 105 may be “in bulk” or in a sample tube, i.e., before or independent of any partitioning of the sample into aqueous compartments. The pre-amplification generates amplicons that include copies of a target of interest, such as a particular variant, including, for example, a structural variant, or SV. Due to the pre-amplification 105, the sample is enriched for the target of interest (relative to other components in the sample). This is useful where the target is present only very small quantities such as a single molecule in a 3, or 10, or 100 mL sample tube. By specifically pre-amplifying 105 the target of interest, the method 101 avoids losing the target to the dead volume involved in dPCR due to stochastic effects when sampling. At this stage, the sample may be partitioned into aqueous compartments. Partitioning into aqueous compartments may use methods known in the art such as the formation of water-in-oil droplets using a microfluidic system or distributing the sample into a multi-well plate.

The method includes extending variant-specific primers 111 to create copies of the amplicons that were generated by the pre-amplification and also copying the copies with tailed primers 107 to form tailed amplicons. The tailed primers may include a 3′ sequence that anneals to the amplicons (i.e., matches or is complementary to the target of interest) and a 5′ tail that offers some functionality. The 5′ tail could include, for example, any one or more of a primer binding site, a sequencing adaptor, a restriction site, a probe binding site, a sample barcode, a unique molecular identifier, an index or tag, others, or any combination thereof. Preferably, the tail of the tailed primers includes at least a universal probe binding site and/or a universal primer binding site. Copying with tailed primers 107 and variant specific primers 111 produces tailed amplicons (that themselves include copies of the specific variant of interest).

The method includes detecting 115 the target of interest by amplifying the tailed amplicons with universal primers and detection probes that anneal to the tailed amplicons. Extension of the universal primers along the tailed amplicons through annealed detection probes generates a signal that shows the presence of the variant in the sample. For example, the detection probes may be fluorescent hydrolysis probes that include a fluorophore and a quencher. When the universal primers are extended by polymerase (to copy the tailed amplicons), the 5′-3′ exonuclease activity of the polymerase digests the fluorescent probe, separating the quencher from the fluorophore, allowing the fluorophore to be detected (e.g., by imaging or fluorescence detection).

Features of the method 101 promote detection of a target nucleic acid of interest (e.g., an SV) by the targeted nucleic acid pre-amplification 105 in an entire sample in a classical PCR format (e.g., single reaction, unpartitioned). Preferably, after preamplification, a DNA polymerase (e.g., Taq polymerase) used in the pre-amplification 105 is inactivated. DNA polymerase enzyme inactivation of the entire pre-amplified sample may proceed using a thermolabile proteinase K. Some embodiments further include the inactivation of the thermolabile proteinase K (e.g., by heating). After the pre-amplification 105 (and any polymerase inactivation and proteinase denaturation), the method 101 preferably includes targeted nucleic acid amplification and detection of a fraction of the pre-amplified and enzyme inactivated sample, using partitioned/compartmentalized PCR (e.g., digital PCR).

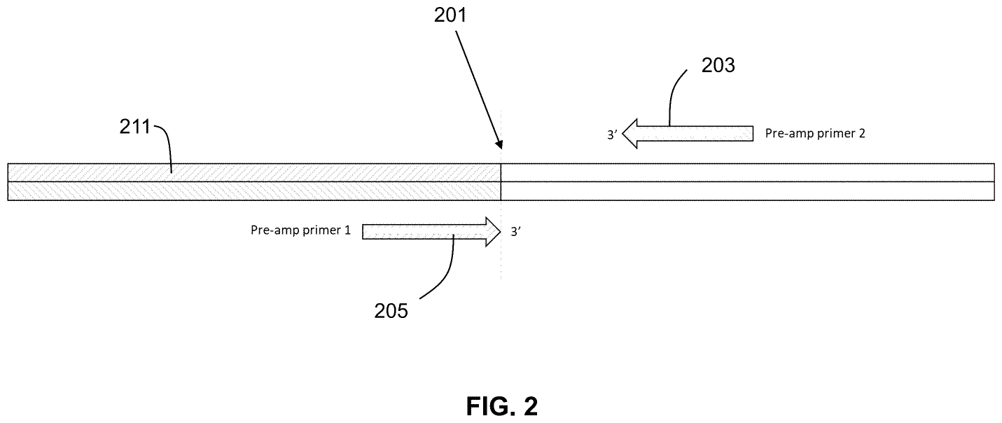

FIG. 2 illustrates elements used in the pre-amplification 105. In general, a pair of primers exponentially amplify a variant of interest 201 (here shown as the breakpoint of a structural variant, or SV) in a classical PCR format (not digital PCR). Here, for example, a first pre-amp primer 205 and a second pre-amp primer 203 anneal to locations on a DNA molecule 211 that flank the variant 201. Specifically, a pair of primers bind to sequences on opposite sides of a target SV breakpoint. The primers may be present in the PCR reaction at relatively low concentrations (50-200 nM). Thermocycling may be performed (e.g., preferably at least about 7 cycles). The reaction mixture includes the input cfDNA 211 containing target variants 201 (e.g., SVs), PCR supermix (DNA polymerase, dNTPs, MgCl2, buffer), first primer 205, and second primer 203. It may be beneficial to use a reduced concentration of PCR supermix compared to standard. For the first primer 205 (Pre-amp primer 1), a majority or entire sequence is a subsequence of the 5′ end of the breakpoint spanning primer (described below). The 3′ end of first primer 205 shares a common sequence with the breakpoint spanning primer, but the 3′ end of the breakpoint spanning primer preferably extends at least approximately 6-8 bases further with an unshared sequence.

For the first primer 205, it may be preferable to use a target melting temperature range of about 48-54° C. A relatively “low” primer concentration may be used in the reaction (e.g., 50-200 nM). For the second primer 203 (“Pre-amp primer 2”), the 3′ end preferably shares approximately 4 bases with the 5′ end of the SV-targeting segment of the tailed primer (discussed below). Preferably, the second primer 203 has a target melting temperature range of about 48-52° C. The second primer 203 may be used at a relatively “low” primer concentration in the reaction (e.g., 50-200 nM).

The pre-amplification 105 includes thermocycling. In some embodiments, the thermocycling involves at least approximately 7 exponential pre-amplification cycles (noting that seven (7) cycles should be equivalent to approximately 128× amplification assuming 100% efficiency and under realistic conditions should achieve at least 10× amplification). The thermocycler may be set to an annealing temperature at which either primer is capable of efficient, but specific, activity (e.g., an annealing temp within the range of about 52-60° C.).

After the pre-amplification, it may be preferable to inactivate or remove the polymerase. Any suitable method may be used to inactivate or remove the polymerase. For example, at least one of the pre-amp primers may be biotinylated allowing the amplicons to be pulled down on magnetic beads and washed. The polymerase may be removed by a lab technique such as size separation, centrifugation, chromatography, or column separation. The products of the pre-amplification 105 may be run out on a gel by polyacrylamide gel electrophoresis and the band corresponding to amplicons may be excised and treated to liberate the amplicons. In a preferred embodiment, the polymerase from the pre-amplification is inactivated, e.g., enzymatically.

In general, DNA polymerase inactivation may be accomplished using a proteinase. For example, residual DNA polymerase present in pre-amplified samples may be inactivated using thermolabile proteinase K prior to detection by digital PCR. In some embodiments, thermolabile proteinase K is added to the samples and incubated at 37° C. for at least 10 minutes. The proteinase K digests and inactivates the polymerase. After polymerase inactivation, the method may include proteinase K inactivation. Thermolabile proteinase K may be inactivated by heat after its use to inactivate residual DNA polymerase and prior to detection digital PCR. In some embodiments, inactivation of proteinase K is performed by incubation at 98° C. for at least 5 min. Thus, in these exemplary embodiments, the method 101 includes inactivating a polymerase enzyme in the sample after the pre-amplification 105.

Inactivating the polymerase with a thermolabile proteinase followed by heat denaturing the proteinase prior to downstream steps provides certain benefits in terms of sample handling, reagent exchange, or clean up (or lack thereof). That is, sample may proceed through a streamlined laboratory handling process with enzyme deactivation, allowing all steps of the method 101 to interoperate and perform as intended, but avoiding the need for various cleanup steps. Preferably, no sample cleanup is performed between steps and the copying or detection step may even be performed in the presence of residual primers and reagents from the pre-amplification.

After pre-amplification and any enzyme inactivation, the method 101 preferably includes detection by digital PCR (dPCR). Specifically, the method may include partitioning the samples into aqueous compartments after the pre-amplification and performing the amplifying step as digital PCR in the aqueous compartments.

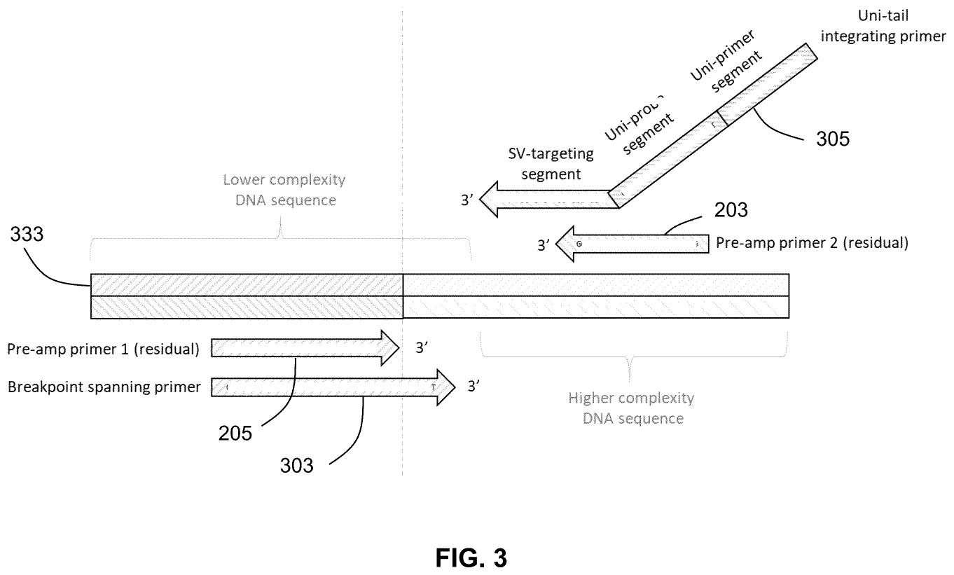

FIG. 3 illustrates elements used in annealing and extending 111 variant-specific primers and copying 107 the copies with tailed primers to form tailed amplicons. It is important to note that while some of these steps are, of necessity, discussed in a certain order, the method 101 is not limited to the recited order. More subtly, the elements of some of the recited steps may be included in the reaction mixture and any given step may proceed when suitable conditions obtain. Thus, the variant specific primer 303 and the tailed primer 305 may be present together in the reaction mixture. When the thermocycler provides the appropriate conditions, the tailed primer 305 anneals to amplicons 333 (from the pre-amplification step) or to a copy made by extending the variant specific primer 303.

The variant specific primer 303 anneals to amplicons 333 (from the pre-amplification step) or to extended tailed primers. Thus, as shown, two steps are happening in the reaction mixture: (1) annealing and extending 111 variant-specific primers to create copies of the amplicon; and (2) copying 107 the copies with tailed primers to form tailed amplicons. That is, progress through the method 101 includes both extending 111 variant-specific primers 303 to create copies of the amplicons that were generated by the pre-amplification and also copying 107 the copies with tailed primers 305 to form tailed amplicons.

The diagram shows a reaction that includes extending 111 variant-specific primers 303 and copying 107 products with a tailed primer 305. The reaction may include input pre-amplified cfDNA containing target structural variants (SVs); PCR supermix (DNA polymerase, dNTPs, MgCl2, buffer); and residual preamplification primers 203 and 205. Here, the variant-specific primers 303 are designed to anneal near, and be extended through, a variant of interest. Preferably, the variant-specific primers are designed to anneal to, and only be extended in the presence of, a variant of interest.

In certain embodiments, the variant comprises a breakpoint of a structural variant (SV) and the variant-specific primers span the breakpoint, and could be described as a breakpoint spanning primer. A breakpoint spanning primer is preferably a high melting temperature (Tm) primer that shares much of the same sequence of first primer 205 but include additional bases in the 3′ direction and must span the breakpoint. Typically, the breakpoint is spanned by approximately 6 bp. The primer 303 is preferentially designed to bind on the lower sequence complexity side of the breakpoint. The 3′ end spans the breakpoint with at least approximately 6 bases. This primer 303 may have a relatively “high” annealing temperature: 58-75° C. and may be provided at typical primer concentrations, e.g., 400-1000 nM.

These steps also use a tailed primer 305. The tailed primer may be used to integrate universal primer and/or probe binding sites into amplicons, and may be referred to as a uni-tail integrating primer. In some embodiments, the uni-tail integrating primer includes three segments: an SV-targeting segment at the 3′ end and a 5′ tail. The 3′ end anneals to one strand of target SV and preferentially is designed to bind on the higher sequence complexity side of the breakpoint. The primer 305 may have an individual segment annealing temp of about 50-58° C. The 5′ tail is preferably specific to custom designed universal primers (Uni-primers). One universal primer is used in a single reaction. The tail integrates a universal primer binding sequence to copies of the original template sequence. The individual segment annealing temp may be about 40-50° C.

As a result of these steps, the method 101 produces an abundance of tailed amplicons in those aqueous compartments that received a copy of the target nucleic acid after the pre-amplification 105 step. Those tailed amplicons are subject to detection by a digital detection reaction with a probe.

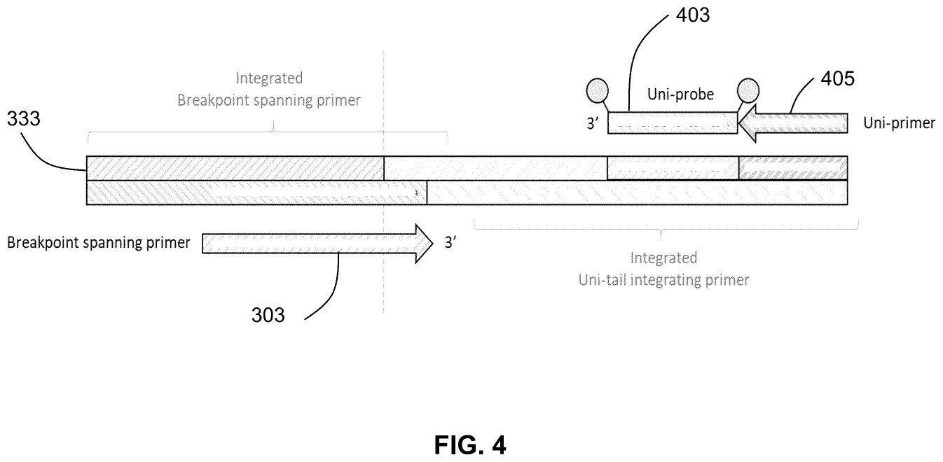

FIG. 4 shows a tailed amplicon 333 being exposed to reagents and conditions that provide for detection 115 of the target (which has been copied into the tailed amplicon 333). Specifically, the detection 115 preferably includes amplifying the tailed amplicons 333 with at least one universal primer 405 (i.e., “universal primers” being plural meaning that a large number of copies of one universal primer 405 may be introduced into the aqueous compartment, where “universal” refers to a sequence designed to anneal to another human-designed and synthetic sequence, as compared to primers designed to uniquely anneal to genomic loci). Preferably, the universal primer 405 is extended by a polymerase in the presence of a detection probe 403 (which may be referred to as “detection probes” to carry the meaning that a large number of copies of one detection probe 403 are present) that anneals to the tailed amplicons 333. Extension of the universal primers 405 along the tailed amplicons 333 allows a 5′-3′ exonuclease activity of the polymerase to digest the detection probe 403. In that sense, the universal primer 405 is extended through the annealed detection probes 403. The probes 403 are digested, which generates a signal that shows the presence of the variant in the sample.

Any suitable probe may be used for the detection probe 403. For example, the detection probe may include an electrochemical label such as ferrocene that can be detected distinctly for digested and un-digested probe using electrodes in the aqueous compartment. In certain embodiments, the detection probes 403 are fluorescent hydrolysis probes such as TaqMan probes from Thermo Fisher Scientific (Waltham, MA). Such fluorescent hydrolysis probes preferably include an oligonucleotide linked to a fluorophore and a quencher. When the probe is intact, the probe does not fluoresce. When the oligonucleotide is digested, the fluorophore is separated from the quencher and fluoresces, which gives a signal that can be detected in various benchtop instruments such as dPCR instruments as well as by using fluorescent microscopy.

Preferred embodiments of the detection probe 403 anneal to a central segment of the Uni-tail integrating primer. Many universal probe segments may be used in a single reaction. The tailed primer integrates a universal probe binding sequence to copies of the original template sequence. The probe 403 may have an individual segment annealing temp of about 50-60° C. and may be used at a concentration of approximately 50-300 nM. The probe concentration may be low compared to Uni-primer concentration, which is in competition for binding/extension due to partial sequence overlap. Preferably the probe is a hydrolysis probe that anneals to the tail and is digested by exonuclease activity of a polymerase used for the amplifying step. In some embodiments, the probe is a fluorescently labelled (5′-end) hydrolysis probe that binds to amplicons once the Uni-probe segment of the Uni-tail integrating primer has been integrated. The probe may include a quencher at 3′-end and optionally may also be internally quenched. The described method and reagents are compatible with the use of multiple fluorescent dyes of different emission spectra. The probe may have an annealing temp: of about 50-60° C. and may be used at a concentration of approximately 250-1000 nM.

The method 101 uses a universal primer 405. The universal primer 405 binds to amplicons once the Uni-primer segment of the Uni-tail integrating primers have been integrated. The universal primer 405 may have an annealing temp of about 40-50° C. and may be used at a concentration approximately of about 500-1500 nM. Concentration of the universal primer 405 may be high compared to Uni-tail integrating primer concentration, which is in competition for binding/extension due to partial sequence overlap.

In some embodiments, activity of the Uni-primer is necessary for signal generation. Activity of the Uni-tail integrating primer is necessary for target specific amplification as well as integration of the “universal sequences”. Activity of the Uni-tail integrating primer must precede activity of the Uni-primer, but more total activity is required of the Uni-tail integrating primer.

In the described method 101, when an aqueous compartment gives a fluorescent signal that is detected, the signal is given because the probe 403 bound to the tailed amplicon 333 and was digested, which means that the tailed amplicon was present, which means that the variant-specific primer 303 bound to the variant. Thus, the signal from the probe 403 shows that the variant (or a copy thereof) was present in the aqueous compartment, meaning that the variant (or other such target of interest) was present in the sample.

By those means the method 101 may be used to detect a target of interest in a sample. The method may be used for any suitable target in any sample. In certain preferred embodiments, the method 101 is used to detect a structural variant (SV) and specifically, the breakpoint of an SV. The breakpoint may have been initially identified by sequencing tumor nucleic acid to obtain tumor-specific sequence reads, or “tumor reads”. The tumor reads may be compared to reference data for a healthy, non-tumor genome. For example, the tumor reads may be compared to reads from a “matched normal” sample (sequence reads obtained by sequencing non-tumor nucleic acid from the subject). In another example, tumor reads are analyzed without the use of “matched normal” reads, by comparing the tumor reads to one or more reference human genomes. For example, tumor reads may be mapped to a reference genome to identify sets of the reads that map to reference genome at positions or orientations that are discordant with how those reads were taken from the tumor nucleic acid.

Where tumor reads map to a reference genome at positions that are inconsistent with loci of those reads in the tumor nucleic acid, the presence of an SV (in the tumor nucleic acid relative to the reference genome) may be inferred. One may design primer pairs that flank breakpoint of the SV and use those as a first pre-amp primer 205 and a second pre-amp primer 203, bringing the workflow up to the pre-amplification 105 step of the method 101. In practice sequencing tumor reads to identify a variant and design primers for use in the method 101 may occur at a different time than performing the method 101.

For example, a sample from a patient with a tumor (e.g., an FFPE slice from a tumor) may be analyzed to design any one or more of the first pre-amp primer 205, the second pre-amp primer 203, and the variant specific primer 303. In parallel, and/or at some other time, the patient may undergo treatment to eradicate the tumor. After the treatment, including for example hours, days, months, and/or years after the treatment, the method 101 may be performed to detect the presence of circulating tumor DNA (ctDNA) from the tumor in the patient's blood or plasma.

One key benefit of this approach is that, after a patient is treated for cancer, follow-on clinical visits can get actionable information with only a minimally-invasive blood draw, i.e., a phlebotomy procedure, sometimes known as a liquid biopsy. That is, after a cancer treatment, continuing follow-up is relatively straightforward and does not require painful procedures or long hospital stays for the patient. Another benefit of the method 101 is that the variant detection can be performed using universal primers 405 and/or universal probes 403. That is, compared to some digital PCR assays that require fluorescent probes to be custom synthesized for every target of interest, here, custom synthesis is only required for simple DNA oligonucleotides. The detectably labeled oligos need not be designed to be complementary to a specific sequence in a genome. Thus, the cost of custom design is applicable only to standard DNA oligos and the cost of fluorescent oligos is applicable only to certain probes that share a “universal” sequence (i.e., common across multiple uses of the method 101, even when the method 101 is applied to multiple patients). The same applies to the universal primer 405, noting further that the use of a universal primer allows full multiplexing (multiple distinct variants within a patient sample and/or multiple patient samples within an instrument run, or even multiple samples over different days in a clinical service laboratory, but always using the same probes and primers for detection and readout).

Those described features of the method 101 provide for the very highly sensitive detection of variants in samples and avoid specific problems of very rare (even single molecule) targets being lost to stochastic phenomena. In methods of the disclosure, pre-amplification product is used as input for subsequent multiplex digital PCR. Digital PCR may be performed using methods described in U.S. Pat. No. 11,066,707, incorporated by reference and hereafter referred to as “SAGAsafe”. Preferably, SAGAsafe compatible PCR is used, but standard exponential PCR is also possible. Since no cleanup is performed between PCR (pre-amp 105) and dPCR (copying and detecting 115), residual preamplification primers, inactivated PCR enzyme, etc., may be carried into the dPCR reaction. The amplifying step may be performed with the universal primers and with the variant-specific primers. Steps of the method may be performed with multiplexed primer and probe sets that amplify and detect multiple distinct variants of interest. Identical copies of the universal probe can be used simultaneously in a single dPCR assay to detect the multiple distinct variants of interest.

The detection assay may be performed as well as, or in parallel to, or multiplexed with, a reference assay. A reference assay may use steps of the method 101 performed with reference primer and probe sets that amplify and detect copy number stable loci to produce a reference signal. With a reference assay, the method may include estimating genomic equivalents in the sample from the reference signal. For a reference assay, primers and probes are included targeting one or more relatively copy number stable regions of the human genome. This enables quantification of haploid human genome equivalents that were not subject to pre-amplification. Primers for a reference assay preferably anneal to a relatively copy number stable region of the human genome, e.g., at an annealing temp: of about 50-60° C. Reference assay component may be used at concentrations of approximately 500-1500 nM for primers and 250-1000 nM for probes. A fluorescent signal detected in an optical channel in the reference assay may be different than those allocated for the detection and quantification of pre-amplified SVs.

In certain embodiments, thermocycling may be either as described in SAGAsafe or classical endpoint PCR. It may be preferable to use SAGAsafe thermocycling program so that SAGAsafe point-mutation detection assays can be multiplex in the same reactions detecting pre-amplified structural variants. SAGAsafe is described in US 2018/0340230, incorporated by reference.

SAGAsafe PCR methods include a linear first stage. During the first stage of the thermocycling program, the annealing/extension temperature is set very high, such that only oligonucleotide components with similarly high melting temperatures are efficiently active. Ideally, in this stage, only the breakpoint spanning primer is efficiently active. If so, the amplification is mostly linear/incremental, with only one of the two SV target strands copied per cycle. Program details for the first stage may include an annealing/extension temperature: 60-72° C. for approximately 32 to 64 cycles.

SAGAsafe PCR methods include an exponential second stage. In the second stage of the thermocycling program, the annealing/extension temperature is set low, such that all oligonucleotide components are efficiently active. In the second stage, active primer components may include pre-amp primers 1 and 2 (e.g., first and second primers 205, 203) in residual amounts present at low concentrations (so activity is possible but limited); the breakpoint spanning primer (e.g., variant specific primer 303) (which was active during “Linear Stage” and continues activity into “Exponential Stage”); uni-tail integrating primer (tailed primer 305); and Uni-primer 405. The tailed primer 305 and the uni-primer 405 are in competition for binding given their partial sequence overlap. The Uni-primer 405 (and Uni-probe 403) can only act on copies derived from the activity of the Uni-tail integrating primer.

The concentrations of competing components are optimized such that high quality signals are generated. A thermocycler may be set with an annealing/extension temperature of 52-62° C. for approximately 27 cycles.

Described methods and reagents provide a number of features and benefits. For example, methods of the disclosure use at least two distinct amplifications.

Digital PCR (dPCR) facilitates absolute quantification of nucleic acids without the need for standard curves, it requires less optimization than quantitative PCR (qPCR) and provides clear discrimination between signal and background noise. A downside to dPCR analysis, however, is the unavoidable dead volume included when using all currently available dPCR systems. This necessarily limits maximum achievable sensitivity. Additionally, maximum allowable sample input volumes per dPCR reaction are typically less than final sample elution volumes of nucleic acid extraction kits. Thus, to maximize sensitivity, a sample is split (or “partitioned”) and analyzed across multiple dPCR reactions (“aqueous compartments”, which may be e.g., droplets or wells). Methods of the disclosure use an initial classical (non-dPCR) nucleic acid amplification (i.e., pre-amplification 105) to overcome the sensitivity disadvantages of dPCR. If the entire sample volume is first sufficiently pre-amplified in a single classical PCR reaction, that sample is protected from false-negatives due to dead volume when used as input to dPCR. Also, the entire sample volume need not be analyzed.

To illustrate, if a sample contains 1 target nucleic acid in 40 uL total volume, then if the sample undergoes classical pre-amplification to 40 copies, the dead volume of the dPCR system is 50%, and ¼th of the pre-amplified sample is analyzed by subsequent dPCR, on average 5 target nucleic acid copies will be detected.

Methods of the disclosure include DNA polymerase enzyme inactivation. Such methods allow the methods to include no intermediate cleanup between classical nucleic acid pre-amplification 105 and dPCR. Such cleanups are tedious, variable in terms of yield, and introduce additional sample loss.

During dPCR reaction setup, pre-amplified sample is added as input. This pre-amplified sample may contain residual, but active, polymerase enzyme. An enzyme inactivation step is used at the conclusion of the classical pre-amplification PCR thermal cycling program, but the heat inactivation is not 100% effective. Therefore, residual polymerase may act to extend primer dimers and other non-specifically bound components. Such polymerase activity generates false-positive producing nucleic acid sequences, particularly those involving the Uni-tail integrating primer since it contains the universal probe binding sequence.

As mentioned above, the methods may include the inactivation of proteinase K prior to digital PCR setup, optionally specifically through the use thermolabile proteinase K. Proteinase K may be used to thoroughly inactivate residual, but active, polymerase present in the sample immediately following classical pre-amplification PCR. Since there need not be any additional post PCR cleanup, unless inactivated, proteinase K present in the pre-amplified sample will act to inhibit the subsequent dPCR reaction.

A thermolabile proteinase K is capable of polymerase inactivation at ambient to moderately warm temperatures and is itself inactivated by incubation at moderate temperatures (55° C.) for a relatively short duration. The procedure is simple, rapid, induces no sample loss, and does not significantly change the sample volume (only ˜1 uL volume added). Non-thermolabile proteinase K (non-thermolabile) is difficult or impractical to heat inactivate using standard laboratory equipment.

Methods of the invention use universal probes. The use of target specific hydrolysis probes results in expensive assays and long development time. Primers can be cheaply and rapidly ordered and delivered from several commercial providers in days. Hydrolysis probes, however, are far more expensive and take weeks to arrive, in particular for fluorophores other than the most commonly used (e.g., FAM, HEX, VIC).

When universal probes are used, large scale batches of probe are ordered and kept in stock which drastically reduces cost and development turnaround time. The use of a relatively small set of universal probes conjugated with alternative fluorescent dyes also enables high multiplexing flexibility without the need to order additional assay components. The universal probes bind to sequences integrated into low-cost, rapidly delivered primers.

The universal probes design strategy is particularly suited for detection of structural variants (SVs). The universal probe design strategy uses primers to detect target specific sequences. The target sequences that are present due to SVs substantially differ from wildtype sequences in just such a fashion that pre-amplification primers are well-suited to amplifying SVs.

The disclosed reagents and methods using universal primers and probes provide more consistent signals than dPCR with sequence-specific primers and probes for each target of interest. Digital PCR assays that use entirely different assay components produce highly variable differences in signal (e.g., cluster dispersion, positive and negative signal amplitudes, noise characteristics). The universal probe design strategy uses multiple common components (Uni-probe, Uni-primer, a segment of the Uni-tail integrating primer). This has resulted in more predictable, better recurring signal patterns, which simplifies assay development, assay validation, and signal thresholding.

Universal primers and probes provide fully discriminatory, multiplex assays. Up to 4 (potentially more) different SVs can be simultaneously tracked and fully discriminated in the same dPCR reaction, together with another locus derived from a relatively copy-number stable region of the human genome used as both internal control and reference.

Preferred embodiments use a high melting temperature variant specific primer 303 that spans a breakpoint of an SV. That is, the primer 303 spans a breakpoint and has a high TM. One of the SV-specific PCR primers designed for each genomic rearrangement stems from the sequence of one of the fusion partners and spans approximately 6 bp into the sequence of the second fusion partner. This strategy enhances the specificity of the approach since it minimizes the extension of the primer at high annealing temperatures unless the fusion sequence is present in the sample.

Certain embodiments use certain rules for the last three nucleotides at the 3′ of the Uni-tail Integrating primer (tailed primer 405). Having the 3′ ends rich in As and Ts minimize risk of primer dimer formation.

The tailed primer 405 preferentially anneals on the high complexity region side of the breakpoint. This strategy minimizes the risk for off-target priming, which may lead to the generation of false positives (i.e., detection of PCR amplicons that are not related to the targeted SV). The 3′ end of the breakpoint-spanning primer adds enough specificity to avoid primer extension even when partly annealed (5′ end) to low complexity DNA sequence regions.

In certain embodiments, the gap between primer pairs is as small as possible. Leaving a maximum of 10 bp between the 3′ end of the breakpoint-spanning primer and the 3′ end of the Uni-tail integrating primer contributes to shorten amplicon size, a critical consideration when it comes to the tracking of highly fragmented ctDNA.

Methods of the disclosure use different primers for pre-amplification and detection PCR. The accumulation of primer dimers and off-target products during preamplification constitutes a source of competition for PCR resources that can detrimentally affect the efficient replication of the targeted SVs. The re-use of the same set of primers for both preamplification and dPCR would increase the probability to generate false positives during the universal probes-based approach as well.

Preferred embodiments use a SAGAsafe program for detection dPCR. The high Tm breakpoint spanning primer is the only primer that can be efficiently extended during the linear phase. This phenomenon promotes the enrichment of partitions on the fusion sequence before the start of the second, exponential phase. Probe hydrolysis is expected to happen only during the exponential phase. Therefore, it is expected to accumulate higher fluorescent signals in partitions carrying the fusion sequence versus those partitions containing off-target products (noise). This phenomenon enables the use of proper thresholding rules to discriminate between “true” and “false” positive partitions.

It is also noted that it may be preferable to preferentially design the Uni-tail Integrating primer with low GC content at the 3′ end (e.g., very few G's and C's in last 3 bases of 3′ end). Preferably, the tailed primer includes only one G or C in the second or third last base. Primers may preferably be designed to be compatible with the SAGAsafe thermocycling program.

Unlike mechanism of PCR using intercalating dyes, here, the use of universal probes does not require a special master mix (e.g., with SYBR Green I, or EvaGreen) and has a more complicated mechanism with additional components beyond target-specific primers (universal primers, universal probes, tailed target-specific primers). While pre-amplification 105 is discussed, it is important to note that the method 101 works without any form of pre-amplification. The pre-amplification is used to increase sensitivity of the assay (primarily by overcoming false-negatives due to dead volume introduced by the dPCR workflow).

What is claimed is:

1. A method comprising:
amplifying a target nucleic acid using tailed primers to form tailed amplicons;
further amplifying the tailed amplicons with universal primers and detection probes that anneal to the tailed amplicons, wherein extension of the universal primers along the tailed amplicons through annealed detection probes generates a signal that shows the presence of a variant in the sample.

2. The method of claim 1, wherein the detection probes are hydrolysis probes.

3. The method of claim 1, wherein the method further comprises performing a pre-amplification prior to the amplifying step.

4. The method of claim 1, wherein the sample is selected from blood and plasma, and the nucleic acid is cell-free DNA (cfDNA).

5. The method of claim 1, wherein the variant is a structural variant (SV) and the amplifying step is performed with a PCR primer pair designed to anneal to sites that flank a breakpoint of the SV.

6. The method of claim 5, wherein a variant-specific primer spans the breakpoint.

7. The method of claim 1, wherein the detection probes comprise a hydrolysis probe that anneals to the tailed amplicons and is digested by exonuclease activity of a polymerase used for the amplifying step.

8. The method of claim 1, further comprising partitioning the samples into aqueous compartments and performing the amplifying step as digital PCR in the aqueous compartments.

9. The method of claim 8, wherein the aqueous compartments are droplets or wells and the detection probe is a fluorescent hydrolysis probe.

10. The method of claim 1, further comprising inactivating a polymerase enzyme in the sample after the pre-amplification.

11. The method of claim 10, wherein the inactivating step is performed with a thermolabile proteinase and the method includes heat denaturing the proteinase prior to the amplifying step.

12. The method of claim 1, wherein no sample cleanup is performed between the pre-amplifying and further amplifying steps and wherein the amplifying step is performed in the presence of residual primers and reagents from the pre-amplification.

13. The method of claim 1, wherein the steps of the method are performed with multiplexed primer and probe sets that amplify and detect multiple distinct variants of interest.

14. The method of claim 13, wherein the detection probes include a universal probe that anneals to a universal probe binding site on a tail of the tailed amplicons.

15. The method of claim 14, wherein identical copies of the universal probe are used simultaneously in a single dPCR assay to detect the multiple distinct variants of interest.

16. The method of claim 1, wherein the steps of the method are performed with reference primer and probe sets that amplify and detect copy number stable loci to produce a reference signal and the method includes estimating genomic equivalents in the sample from the reference signal.

17. The method of claim 1, wherein the generating step comprises amplifying nucleic acid comprising a variant.

18. The method of claim 1, wherein the detection probes are universal probes that anneal to a universal probe binding site on a tail of the tailed amplicons.

19. The method of claim 1, wherein the variant-specific primers are designed to anneal near, and be extended through, a variant of interest.

20. The method of claim 1, wherein the variant-specific primers are designed to anneal to, and only be extended in the presence of, a variant of interest.

21. The method of claim 1, wherein the variant comprises a polymorphism, a small indel, or a breakpoint of a structural variant.

22. The method of claim 1, wherein a universal probe is used to detect and quantify a reference locus.
